(19) United States
(12) Patent Application Publication
(10) Pub. No.: US 2025/0134509 A1
(43) Pub. Date: May 1, 2025

(54) ATRIAL RETRACTOR

(71) Applicant: Scanlan International, Inc., St. Paul, MN (US)

(72) Inventors: Nelson J. Paulo, Gulpilhares (PT); Samuel R. Westlake, Farmington, MN (US); Robert E. Kohler, Lake Elmo, MN (US)

(21) Appl. No.: 18/918,660

(22) Filed: Oct. 17, 2024

Related U.S. Application Data
(60) Provisional application No. 63/593,165, filed on Oct. 25, 2023.

(51) Int. Cl. A61B 17/02; A61B 17/00; A61B 90/00

(52) U.S. Cl. CPC A61B 17/02; A61B 90/361; A61B 2017/00477; A61B 2017/00862; A61B 2017/0237

(57) ABSTRACT

A surgical retractor comprises a handle having a distal end portion defining a distal end axis and a retractor head on the distal end portion of the handle. The retractor head includes a retractor surface portion that forms an acute angle that is substantially less than 90° with respect to the distal end axis. In embodiments, the retractor surface portion is at an angle sufficiently close to parallel with respect to the distal end axis to facilitate insertion into a heart chamber of a patient from a side of the patient in a medial direction aligned with the distal end axis of the handle. Embodiments may comprise an attachment structure on the distal end portion of the handle configured to releasably attach a foil to the retractor head.

### REFERENCE TO RELATED APPLICATION

This application claims the benefit of U.S. Provisional Application No. 63/593,165 filed on Oct. 25, 2023, and entitled Atrial Retractor, which is incorporated herein by reference in its entirety and for all purposes.

### FIELD

This disclosure relates generally to retractor instruments used to retract tissue during surgical procedures. Embodiments include retractors configured to retract tissue in a heart chamber, such as an atrium.

### BACKGROUND

Instruments used to retract tissue during surgical procedures are generally known. The Poo et al. U.S. Pat. No. 8,388,525, for example, discloses an atrial lift retractor and method for retracting tissue in an atrium of a patient's heart during surgical procedures involving the mitral valve or the tricuspid valve.

There remains, however, a need for improved surgical retractors. For example, there is a need for improved retractors capable of enhancing the efficacy and outcomes of surgical procedures on heart structures such as mitral valves and tricuspid valves.

### SUMMARY

Improved surgical retractor instruments and associated methods are described herein. The retractors and methods can provide enhanced patient outcomes, for example when used in connection with any of a range of surgical procedures. Non-limiting examples of surgical procedures that may benefit from the instruments include procedures involving mitral and tricuspid valves, and breast reconstruction procedures.

One example is a surgical retractor. Embodiments of the surgical retractor comprise: a handle having a distal end portion defining a distal end axis; and a retractor head including a retractor surface portion on the distal end portion of the handle, wherein the retractor surface portion forms an acute angle that is substantially less than 90° with respect to the distal end axis.

In some embodiments of the surgical retractor, the retractor surface portion forms an angle that is less than 30° with respect to the distal end axis.

In any or all embodiments of the surgical retractor, the retractor surface portion forms an angle that is less than 15° with respect to the distal end axis.

In any or all embodiments of the surgical retractor, the retractor surface portion forms an angle that is less than 5° with respect to the distal end axis.

In any or all embodiments of the surgical retractor, the retractor surface portion is parallel to the distal end axis.

In any or all embodiments of the surgical retractor, the retractor surface portion is configured at an angle sufficiently close to parallel with respect to the distal end axis to facilitate insertion of the distal end portion of the handle into a heart chamber of a patient, optionally a left atrium or right atrium of the patient, from a side of the patient in a medial direction aligned with the distal end axis of the handle.

Any or all embodiments of the surgical retractor may further comprise an attachment structure on the distal end portion of the handle configured to releasably attach a foil to the retractor head, wherein the retractor head and/or the attachment structure are configured such that a surface portion of the foil, when attached to the retractor head, forms an acute angle that is substantially less than 90° with respect to the distal end axis. For example, the retractor head and/or the attachment structure may be configured such that a surface portion of the foil, when attached to the retractor head, forms an angle that is less than 30° with respect to the distal end axis. For example, the retractor head and/or the attachment structure may be configured such that a surface portion of the foil, when attached to the retractor head, forms an angle that is less than 15° with respect to the distal end axis. For example, the retractor head and/or the attachment structure may be configured such that a surface portion of the foil, when attached to the retractor head, forms an angle that is less than 5° with respect to the distal end axis. For example, the retractor head and/or the attachment structure may be configured such that a surface portion of the foil, when attached to the retractor head, is parallel to the distal end axis. For example, the retractor head and/or attachment structure may be configured such that the surface portion of the foil, when attached to the retractor head, is at an angle sufficiently close to parallel with respect to the distal end axis to facilitate insertion of the distal end portion of the handle and the foil into a heart chamber of a patient, optionally a left atrium or right atrium of the patient, from a side of the patient in a medial direction aligned with the distal end axis of the handle.

In any or all embodiments of the surgical retractor, the attachment structure may comprise a tab.

In any or all embodiments of the surgical retractor, the retractor head includes a first blade; and the attachment structure comprises a second blade adjacent to the first blade, wherein the first and second blades define a slot configured to releasably receive the foil. For example, one of the first and second blades, optionally the first blade, may include a tab extending into the slot. In embodiments, for example, the other of the first and second blades, optionally the second blade, may include an opening; and the tab extends into the opening.

Any or all embodiments of the surgical retractor may further comprise a foil attached to the distal end portion of the handle by the attachment structure, wherein the foil includes a foil opening and the tab extends into the foil opening.

Any or all embodiments of the surgical retractor may further comprise a foil attached to the distal end portion of the handle by the attachment structure.

In any or all embodiments of the surgical retractor, the foil may comprise a resilient member configured to extend between a closed or reduced diameter delivery configuration and an increased diameter open configuration.

Any or all embodiments of the surgical retractor may further comprise a light or imaging device on the distal end portion of the handle. For example, the light or imaging device may define an optical axis that extends at a non-zero acute angle with respect to the distal end axis. For example, the retractor may further comprise one or more optical fibers, each coupled to one of the light or imaging device; and the distal end portion of the handle comprises a port to receive the optical fiber.

In any or all embodiments of the surgical retractor, the port may define a non-zero acute angle with respect to the distal end axis.

Any or all embodiments of the surgical retractor may further comprise one or more ports extending from the distal end portion of the handle.

Any or all embodiments of the surgical retractor may further comprise an optical device mounted in one or more of the one or more ports. For example, at least one of the one or more ports may define a non-zero acute angle with respect to the distal end axis. For example, the retractor my further comprise an optical device mounted in the at least one of the one or more ports defining the non-zero acute angle with respect to the distal end axis. For example, the optical device may comprise a light or a camera.

In any or all embodiments of the surgical retractor, the distal end portion of the handle may be linear; and the linear distal end portion of the handle has a length that is sufficiently long that the linear distal end portion can extend outside of the patient after the distal end portion and the retractor head are inserted into the patient's heart chamber.

Another example is a surgical access method. Embodiments include providing surgical access to a heart chamber of a patient, optionally a right atrium or a left atrium, by a retractor including a handle having a distal end portion defining a distal end axis and a retractor head on the distal end portion, comprising inserting the retractor head into the heart chamber of the patient from a side of the patient at least substantially in a medial direction aligned (e.g., colinear) with the distal end axis of the distal end portion of the handle.

Embodiments of the method may, for example, use a surgical retractor in accordance with any or all embodiments of the surgical retractor example described above, without the foil.

Embodiments of the method may, for example, use a surgical retractor in accordance with any or all embodiments of the surgical retractor example described above, including the foil in the delivery configuration. The method may further comprise causing the foil to open to an operative position in the heart chamber and retract tissue in the heart.

In any or all embodiments of the method, inserting the retractor head includes moving the distal end portion of the handle in a direction parallel to the distal end axis. For example, inserting the retractor head may include inserting a linear portion of the handle including the distal end portion into the patient and positioning the retractor head and optionally the foil in the heart chamber while the linear portion of the handle extends outside of the side of the patient.

### BRIEF DESCRIPTION OF THE DRAWINGS

FIG. 1A is a perspective illustration of a retractor including a handle and a retractor head in accordance with embodiments, generally a showing a first side of the handle and retractor head.

FIG. 1B is a side view of the retractor shown in FIG. 1A, in accordance with embodiments.

FIG. 1C a top view of the retractor, generally showing the side shown in FIG. 1A, in accordance with embodiments.

FIG. 1D is a bottom view of the retractor, showing the side opposite the side shown in FIG. 1C, in accordance with embodiments.

FIG. 1E is an end view of the retractor, showing an end of the retractor head and attachment structure, in accordance with embodiments.

FIG. 2A is a perspective illustration of the retractor shown in FIG. 1A, including a foil attached to the handle, in accordance with embodiments.

FIG. 2B is a perspective illustration of the retractor with the foil shown in FIG. 2A, generally showing a second side of the retractor and foil opposite the side shown in FIG. 2A, in accordance with embodiments.

FIG. 3A is a detailed sectional side view of a distal end of the handle, showing the retractor head and an attachment structure, in accordance with embodiments.

FIG. 3B is the view of the distal end of the handle shown in FIG. 3A, showing also a portion of a foil attached to the retractor head by the attachment structure, in accordance with embodiments.

FIG. 4 is a detailed top view of the distal end of the handle and retractor head, in accordance with embodiments.

FIG. 5A is a perspective illustration of the foil when in the open configuration, generally showing the first side of the foil, in accordance with embodiments.

FIG. 5B is a top view of the foil when in the open configuration shown in FIG. 5A, showing the first side, in accordance with embodiments.

FIG. 5C is an end view of the foil when in the open configuration shown in FIG. 5A, in accordance with embodiments.

FIG. 6A is a perspective illustration of the foil when in the delivery configuration, in accordance with embodiments.

FIG. 6B is a top view of the foil when in the delivery configuration shown in FIG. 6A, in accordance with embodiments.

FIG. 6C is an end view of the foil when in the delivery configuration shown in FIG. 6A, in accordance with embodiments.

FIG. 7A is a detailed perspective illustration of the distal end portion of the retractor, generally showing a first side of the retractor head and a foil attached to the retractor head, in accordance with embodiments.

FIG. 7B is a detailed perspective illustration of the distal end portion of the retractor shown in FIG. 7A, generally showing a second side of the retractor head and foil, in accordance with embodiments.

FIG. 7C is a detailed side view of the distal end portion of the retractor shown in FIG. 7A, showing the retractor head and foil attached thereto, in accordance with embodiments.

FIG. 7D is a detailed end view of the distal end portion of the retractor shown in FIG. 7A, showing the end with the foil attached to the retractor head, in accordance with embodiments.

FIG. 8 is a detailed sectional side view of the retractor head and attachment structure, showing an optical fiber in a tool port, in accordance with embodiments.

FIG. 9A is a diagrammatic illustration of an anterior (e.g., chest) side of a torso of a patient and the patient's heart, and the retractor, with the foil in the delivery configuration, being inserted into the torso by a medial approach from the right side of the patient, in accordance with embodiments.

FIG. 9B is the diagrammatic illustration of the anterior side of the torso and heart as shown in FIG. 9A, with the retractor, and the foil in the delivery configuration, inserted into a chamber of the heart by the medial approach, in accordance with embodiments.

FIG. 9C is the diagrammatic illustration of the anterior side of the torso and heart as shown in FIG. 9B, with the foil deployed to an operative configuration.

FIG. 10A is a diagrammatic transverse plane sectional view of a torso of a patient and the patient's heart, shown from the superior (e.g., head) side, illustrating an access port in the right side of the torso though which a retractor can be inserted into the patient by the medial approach, in accordance with embodiments.

FIG. 10B is the diagrammatic transverse plane view of the torso, heart and access port shown in FIG. 10A, with the retractor, and foil in the delivery configuration, inserted into a chamber of the heart by the medial approach, in accordance with embodiments.

FIG. 10C is the diagrammatic transverse plane view of the torso, heart and access port shown in FIG. 10B, with the foil deployed to the operative configuration.

### DETAILED DESCRIPTION

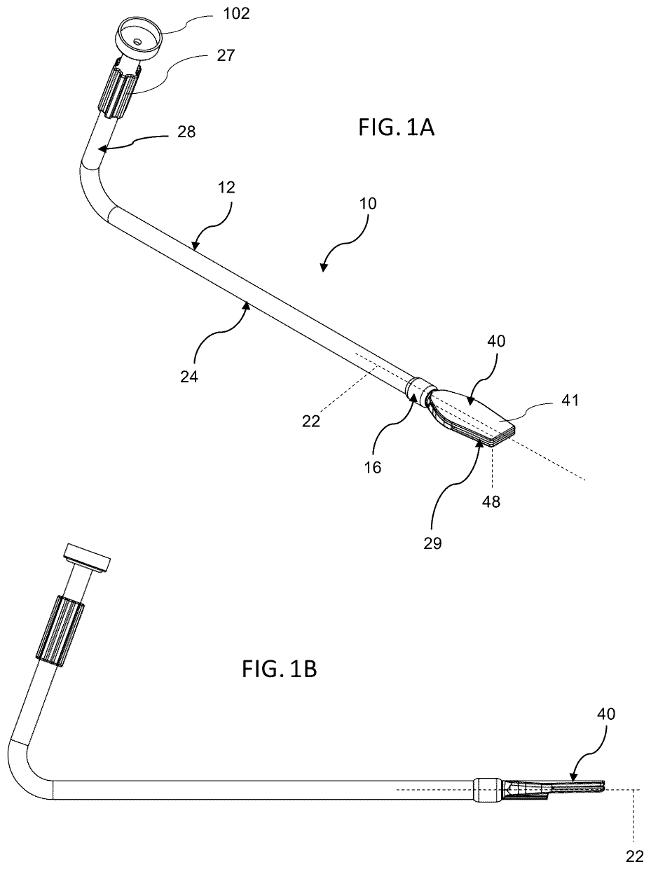

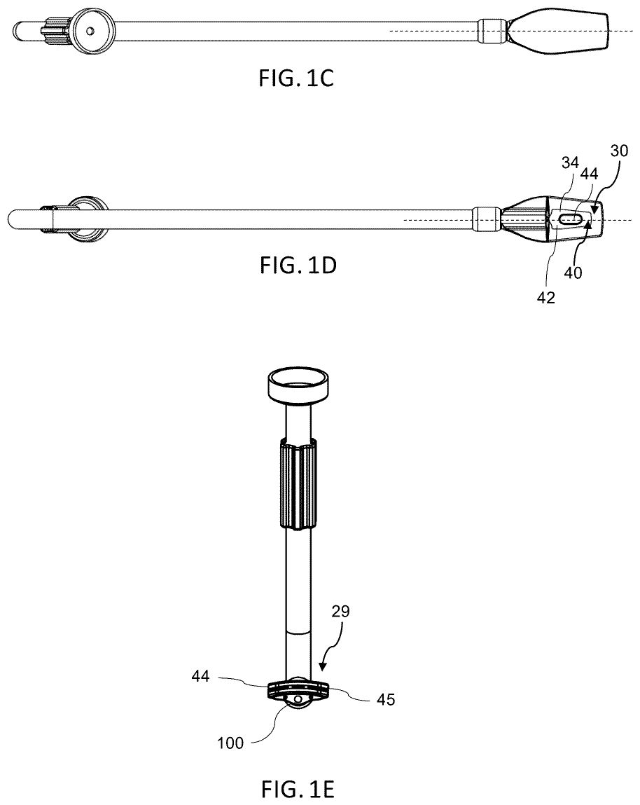

A surgical retractor 10 in accordance with certain embodiments can be described generally with reference to FIGS. 1A-1E. The illustrated embodiments of the retractor 10 include a handle 12 and a retractor head 40 on a distal end portion 16 of the handle. As described in greater detail below, retractor 10 is configured to facilitate direct or in-line medial access of the retractor head 40 into a heart chamber of a patient. By this approach, the retractor 10 is manipulated to locate and position the retractor head 40 into a heart chamber, such as for example a right atrium or a left atrium of the heart. The retractor head 40 thereby urges or retracts certain tissues in the heart chamber away from a surgical site, to provide operative access to other tissues such as for example a tricuspid or mitral valve.

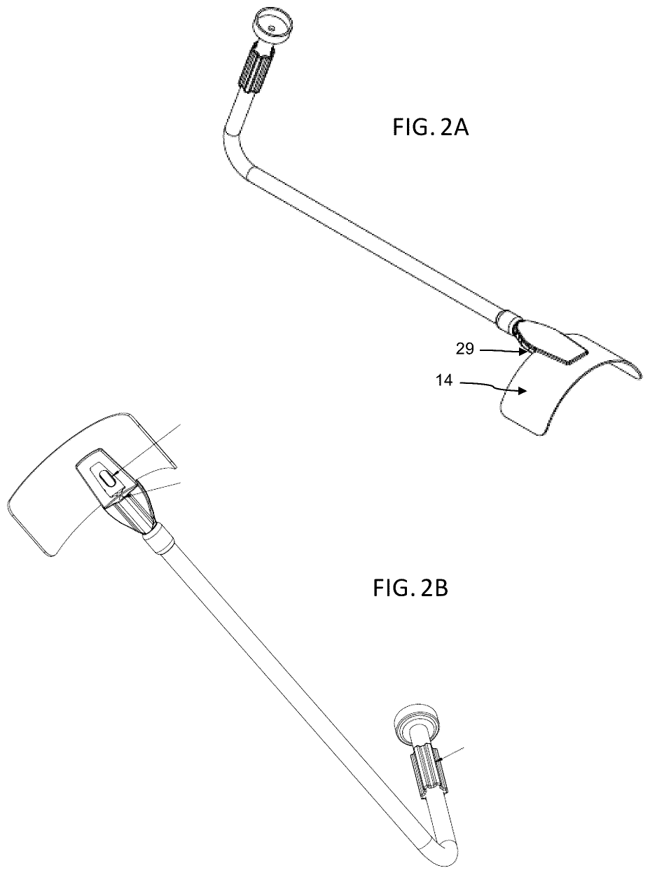

As shown for example in FIGS. 2A and 2B, embodiments of the retractor 10 may optionally also be configured for use with a foil 14. To facilitate the use of retractor 10 with the foil 14, the retractor may include an attachment structure 29 to releasably attach the foil to the distal end portion 16 of the handle 12. In the illustrated embodiments, for example, the attachment structure 29 releasably attaches the foil 14 to the retractor head 40. The foil 14 is shown in an open configuration in FIGS. 2A and 2B, but as described in greater detail below, can be collapsed or otherwise moved to a closed or delivery configuration. When the foil 14 is in the delivery configuration, surgical retractor 10 can be manipulated to deliver, locate and position the foil 14 into the heart chamber of a patient. After the foil 14 is positioned within the heart chamber, it can be released or otherwise deployed to an operative configuration to urge or retract certain tissues in the heart chamber away from a surgical site. Because the foil 14 has a surface that is larger than the surface of the retractor head 40, it may retract more tissue than the retractor head 40 alone, and may enhance the operative access provided by the retractor 10. As described in greater detail below, embodiments of the retractor 10 with the foil 14 are configured to facilitate direct or in-line medial access of the foil 14 into the heart chamber.

As shown for example in FIGS. 1A-1E, 2A and 2B, the distal end portion 16 of the handle 12 defines a distal end axis 22. The distal end portion 16 is linear in the illustrated embodiments, and the distal end axis 22 is colinear with the distal end portion in these embodiments. The handle 12 may include a middle portion 24 extending proximally from the distal end portion 16. Middle portion 24 is linear, and colinear with the distal end portion 16 in the illustrated embodiments. A proximal end portion 28 of the handle 12 extends from the middle portion 24, opposite the middle portion from the distal end portion 16. Proximal end portion 28 of the handle 12 extends at an angle with respect to the middle portion 24, and includes a hand grip 27 in the illustrated embodiments. In general, the distal end portion 16, middle portion 24 and proximal end portion 28 of the handle 12 are generally co-planer in the illustrated embodiments. Other embodiments of the handle 12 have other forms and/or features. For example all or portions of one or both of the distal end portion 16 or the middle portion 24 may be curved.

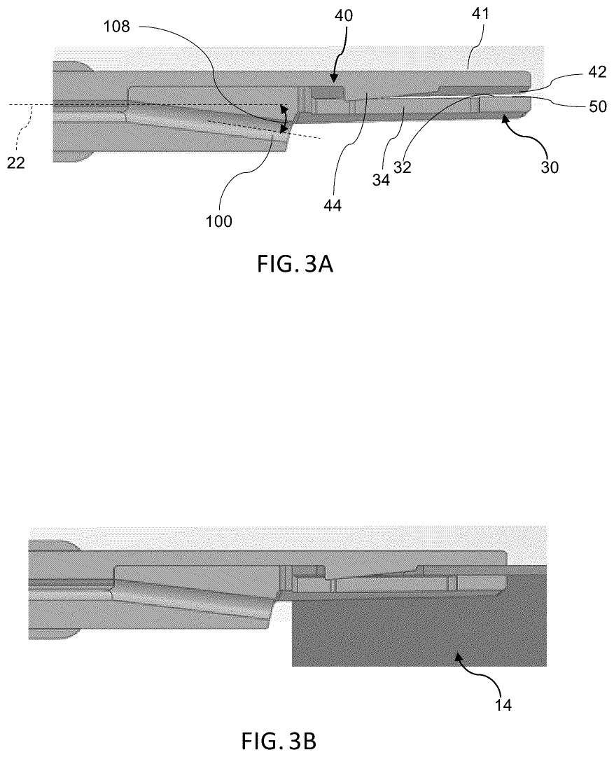

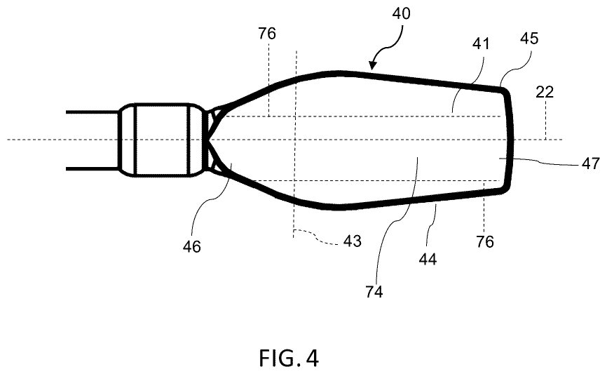

Referring also to FIGS. 3A, 3B and 4, the retractor head 40 is shown as a blade-shaped member that includes a first surface 41 and a second opposite surface 42. The first surface 41 is configured to engage an inside wall of a heart chamber in which the retractor 10 is being used, and is a generally planar surface in the illustrated embodiments. As perhaps best shown in FIGS. 1E and 4, the illustrated embodiments of the first surface 41 is curved about a transverse axis 43 that extends between the opposite side portions 44 and 45 of the retractor head 40. The first surface 41 is thereby convex in the illustrated embodiments, and may conform generally to the inside (e.g., concave) surface of the heart chamber that the retractor 10 is being used to retract. The first surface 41 of the retractor head 40 includes a proximal end portion 46 adjacent to the distal end portion 16 of the handle 12, and a distal end portion 47. The first surface 41 of the retractor head 40 is defined by engagement axes such as 48 that extend between the proximal end portion 46 and distal end portion 47.

An important feature of the retractor 10 is its ability to be used to deliver and position the retractor head 40 into a patient's heart chamber by an in-line medial approach from a side of the patient. By this method the retractor head 40 is positioned and located in the patient's heart chamber through motion of the handle 12 substantially in a direction that is aligned with and parallel to (e.g., colinear with) the distal end axis 22 of the handle. To facilitate this delivery method and use, in the illustrated embodiments the first surface 41 of the retractor head 40, for example as defined by the engagement axes 48, is oriented parallel to the distal end axis 22 of the handle 12. The distal end portion 47 of the retractor head 40 thereby leads during the delivery and positioning of the retractor head in the patient's heart chamber as the handle 12 is moved in a direction parallel to the distal end axis 22.

Although the first surface 41 of the retractor head 40 is preferably oriented substantially parallel with respect to the distal end axis 22 of the handle 12 (e.g., within ±3° of one other), the efficacy and advantages of the retractor 10 may be provided by embodiments that include other angular orientations between the distal end axis 22 and the first surface 41 of the retractor head. For example, in some embodiments the first surface 41 of the retractor head 40 defines an angle of ±5° with respect to the distal end axis 22 of the handle 12. In other embodiments, the first surface 41 of the retractor head 40 defines an angle of ±15° with respect to the distal end axis 22 of the handle 12. In yet other embodiments, the first surface 41 of the retractor head 40 defines an angle of ±30° with respect to the distal end axis 22 of the handle 12. The angle defined between the first surface 41 of the retractor head 40 and the distal end axis 22 of the handle 12 may depend on factors such as personal preferences of clinicians using the retractor head. For example the efficacy and advantages of the retractor 10 may be provided by embodiments where the surface 41 of the retractor head 40 and the distal end axis 22 of the handle 12 define an acute angle that is substantially less than ±90°.

The attachment structure 29 that may be used to releasably attach the foil 14 to the handle 12 can be described with reference to FIGS. 3A and 3B. In the illustrated embodiments the attachment structure 29 includes a member 30 that cooperates with the retractor head 40 to releasably attach and support the foil 14 on the distal end portion 16 of the handle 12. The member 30 is shown as a blade-shaped member in the illustrated embodiments and includes a generally planar surface 32 that faces the second surface 42 of the retractor head 40. The surface 32 of the member 30 and the surface 42 of the retractor head 40 define a slot 50 configured to receive the foil 14, and thereby effectively attach the foil to the retractor head at the distal end portion 16 of the handle 12. The illustrated embodiments of the retractor head 40 and attachment structure 29 also include a tab 44 that extends from the surface 42 of the retractor head toward the member 30. As perhaps best shown in FIGS. 3A and 3B, a surface of the tab 44 facing the member 30 may slope toward the member 30 with increasing distance from the distal end of the retractor 10 (e.g. from the open end of the slot 50). The illustrated embodiments of the member 30 include a recess, shown for example as an aperture 34, that encompasses at least portions of an outer profile of the tab 44 in the illustrated embodiments. The aperture 34 and tab 44 may be configured so that at least portions of the tab extend into the aperture.

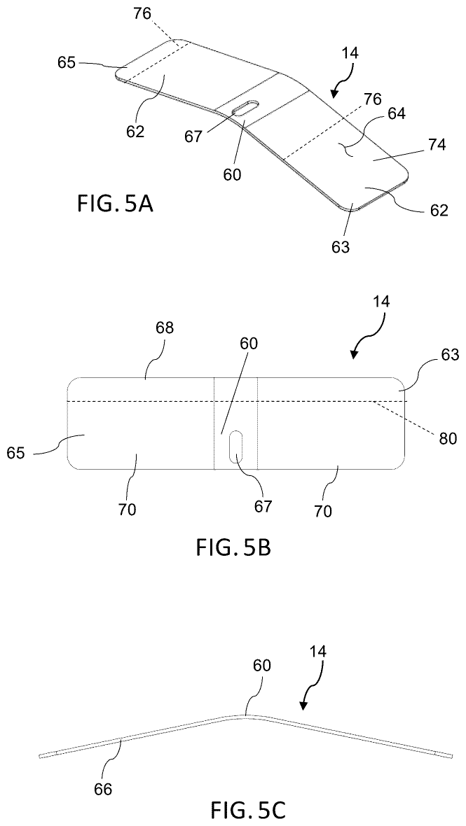

FIGS. 5A-5C illustrate certain features of the foil 14 in its open configuration when no external forces are acting on the foil (e.g., when the foil is in its neutral or free state). As shown, the foil 14 include a mounting portion 60 that is configured to releasably engage and mount to the attachment structure 29, and one or more wings or extending portions 62 that extend transversely from sides of the mounting portion 60. Two extending portions 62 are shown for purposes of example in the illustrated embodiments, and define opposite side portions 63 and 65 of the foil 14. The illustrated embodiments of the foil 14 also include an aperture 67 in the mounting portion 60 that is configured (e.g., sized and shaped) to cooperate with the tab 44 of the attachment structure 29 in the embodiments that include such a tab. Other embodiments of the foil 14 may not include an aperture 67.

A transverse axis 80 extending between the first and second side portions 63 and 65 defines a first diameter of the foil 14 when in the open configuration. The mounting portion 60 and each of the extending portions 62 have a first side surface 64, a second side surface 66, a distal end portion 68 and a proximal end portion 70. As described in greater detail below, the first side surface 64 of the foil 14 is configured to engage the inner surface of a heart chamber of the patient when used on the retractor 10. Engagement portions 74 of the first side surface 64 of the foil 14 are defined by engagement axes such as 76 that extend between the distal end portion 68 and the proximal end portion 70 of the foil. In the illustrated embodiments, the foil 14 is a relatively thin and constant thickness member. The engagement portions 74 on the first side surface 64, and corresponding portions on the second side surface 66, are generally planar in the illustrated embodiments.

In the illustrated embodiments, when in the open configuration, the foil 14 is curved along the transverse axis 80 that extends between the side portions 63 and 65. The open configuration of foil 14 therefore has a curved shape in the illustrated embodiments, with the first side surface 64 having a generally convex shape and the second side surface having a generally concave shape. Other embodiments of the foil 14 (not shown) have other shapes and configurations. For example, the engagement portions 74 may have textured and/or curved portions, and/or the foil 14 may be generally flat in its open configuration.

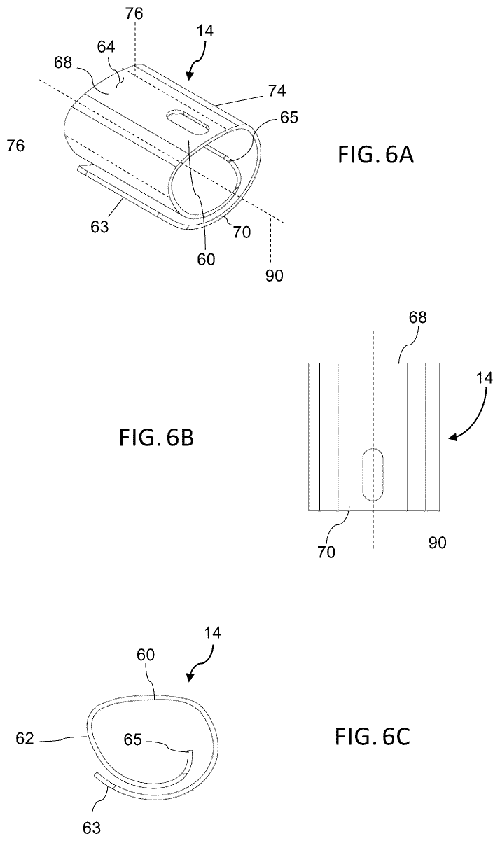

FIGS. 6A-6C illustrate the foil 14 in the closed or delivery configuration. As shown, when in the delivery configuration the first and second side portions 63 and 65 are moved closer to one another than in the open configuration, thereby also moving the two engagement portions 62 toward one other, with the first side surface 64 located on the outside of the collapsed foil 14. The collapsed foil 14 is shown as having a generally tubular shape in the illustrated embodiments (e.g. portions are rolled with respect to one other), although it may have other shapes in other embodiments (not shown). For example the engagement portions 62 may fold, for example toward the mounting portion 60, in such other embodiments. When in the delivery configuration the foil 14 defines a second diameter that is less than the first diameter of the foil when in its open position (e.g., is in a reduced-diameter state), and a longitudinal axis such as the axis 90 shown in FIGS. 6A and 6B that extends between the distal end portion 68 and proximal end portion 70. The longitudinal axis 90 of the foil 14 in the delivery configuration is generally parallel to the engagement axes 76 of the engagement portions 74.

Embodiments of the foil 14 may be formed from resilient materials such as polymer or metals such as shape metals (e.g., nitinol). In embodiments of these types, the foil 14 is manipulated from the open configuration to the delivery configuration against the resilient bias forces of the material, and retained in the delivery configuration while the foil is being positioned within the heart chamber. As described in greater detail below, in embodiments a forceps or other clamp-type instrument or tool can be used to hold, constrain or retain the foil 14 in the delivery configuration while is it being positioned in the patient. Other embodiments the foil may include structures that releasably engage one another to hold the foil 14 in the delivery configuration until they are actuated to release (not shown). In embodiments of these types, the properties of the material of the foil 14 will cause the foil to move toward the open configuration when released from the delivery configuration.

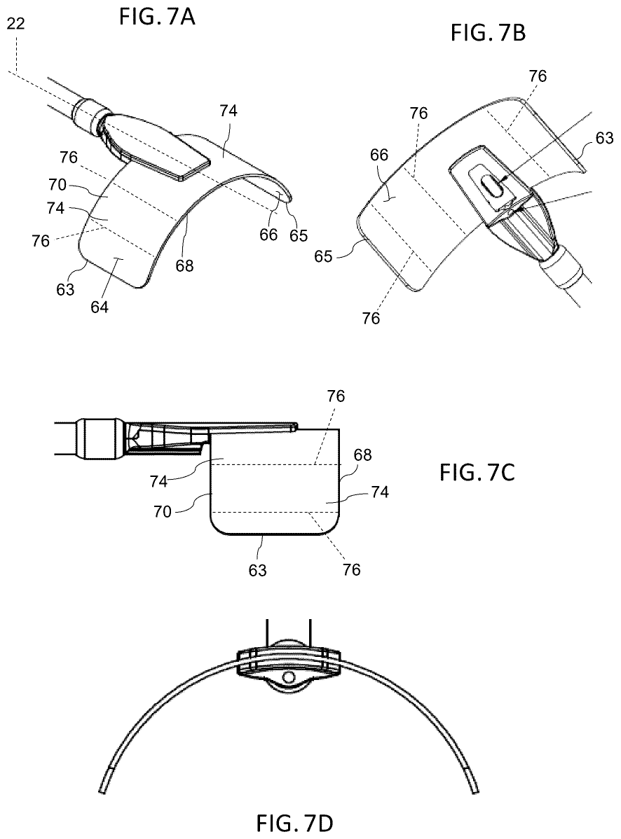

FIGS. 7A-7D illustrate the distal end portion 16 of the handle 12 when the foil 14 is attached to the handle and in the open configuration. As described below, an important feature of the retractor 10 is its ability to be used to deliver and position the foil 14 into a patient's heart chamber (when in the delivery configuration) by an in-line medial approach from a side of the patient. By this method the foil 14 is positioned and located in the patient's heart chamber through motion of the handle 12 in a direction that is substantially aligned with and parallel to (e.g., colinear with) the distal end axis 22 of the handle. To facilitate this delivery method, in the illustrated embodiments the attachment structure 29 is configured to receive the foil 14, and to releasably attach the foil to the handle 12, with the engagement axes 76 of the engagement surface portions 74 oriented parallel to the distal end axis 22 of the handle. The surface portions 74 of the foil 14 extending between the distal and proximal end portions 68 and 70, and the longitudinal axis 90 of the foil when in the delivery configuration, are therefore generally parallel to the distal end axis 22 of the handle 12. The distal end portions 68 of the foil 14 in the delivery configuration thereby lead during the entry and positioning of the foil 14 into the patient's heart chamber as the handle 12 is moved in a direction parallel to the distal end axis 22.

Although the engagement surface portions 74 of the foil 14 are preferably oriented substantially parallel with respect to the distal end axis 22 of the handle 12 (e.g., within ±3° of one other), the efficacy and advantages of the retractor 10 when used with the foil 14 may be provided by embodiments that include other angular orientations between the distal end axis 22 and the engagement surface portions 74 of the foil 14. For example, in some embodiments the first surface portions 74 and/or the longitudinal axis 90 of the foil 14 in the delivery configuration define an angle of ±5° with respect to the distal end axis 22 of the handle 12. In other embodiments, the first surface portions 74 and/or the longitudinal axis 90 of the foil 14 in the delivery configuration define an angle of ±15° with respect to the distal end axis 22 of the handle 12. In yet other embodiments, the first surface portions 74 and/or the longitudinal axis 90 of the foil 14 in the delivery configuration define an angle of ±30° with respect to the distal end axis 22 of the handle 12. The angle defined between the first surface portions 74 and/or the longitudinal axis 90 of the foil 14 in the delivery configuration may depend on factors such as personal preferences of clinicians using the retractor 10. For example, the efficacy and advantages of the retractor 10 may be provided by embodiments where the first surface portions 74 and/or the longitudinal axis 90 of the foil 14 in the delivery configuration define an acute angle with respect to the distal end axis 22 of the handle 12 that is substantially less than ±90°.

As shown for example in FIGS. 1E and 3A, embodiments of the retractor 10 may include one or more tool ports 100 on the retractor head 40 (one is shown for purposes of example) having a distal end that opens on a side of the retractor head opposite the first surface 41 configured to engage the heart chamber during use. The one or more tool ports 100 open also opposite the first side surface 64 of the foil 14 configured to engage the heart chamber during use of embodiments of the retractor 10 with the foil attached thereto. The tool ports 100 therefore provide access to tools such as cameras, lights or suction sources to the surgical site, opposite the retractor head 40 and/or foil 14 from the retracted portions of the heart chamber. The tool ports 100 may extend through the handle 12, for example to the proximal end portion 28 of the handle as shown in FIG. 1A, and may include a coupler such as 102 to couple the tool port and/or a tool mounted therein to an external device such as a source of light, an imaging device for displaying an image received by a camera, or a source of suction for aspiration of tissue and/or fluid from the operative site.

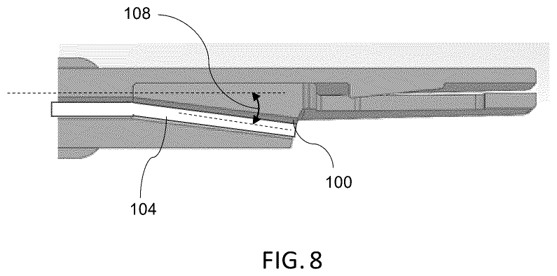

As an example of the types of tools that can be used in connection with the tool port 100, FIG. 8, illustrates an optical fiber 104 mounted within the tool port. The optical fiber 104 can, for example, function as a light source and provide illumination light to the operative site when coupled to a source of light (not shown), or as an imaging device that couples an image of the operative site to an imaging system (not shown). In embodiments, for example, the distal end of the optical fiber 104 adjacent to the retractor head 40 configured to operate as an illumination source or a camera, and may include a lens (not shown). In other embodiments (not shown) a camera or source of light such as an LED may be mounted at the end of the tool port 100 adjacent to the retractor head 40, and other structures such as power supply or optical signal transmission wires that couple to the camera or source of light may extend through the tool port (e.g., to the coupler 102.

In the embodiments shown in FIG. 3A and FIG. 8, the distal end of the tool port 100 opens at a location offset from the retractor head 40 and the foil 14 (when attached) (e.g., in an offset direction generally perpendicular to the distal end axis 22), and at an angle 108 with respect to the distal end axis 22. In embodiments, for example, the angle 108 may be between 3° and 10°. In other embodiments the angle 108 may between 10° and 20°. In still other embodiments the angle 108 may be between 20° and 45°. Other embodiments have a tool port 100 configured at other greater or lesser angles 108 with respect to the distal end axis 22 of the handle 12 (including for example angles of 0° and angles less than 0°. In generally, the amount of positional offset and the angle 108 may depend on factors such as the structure and functionality of the particular tool being used in connection with the tool port 100, and can be selected to optimize the functionality in view of the structure.

When the foil 14 is being used with the retractor 10, the foil is attached to the distal end portion 16 of the handle 12 by the attachment structure 29, and set to its delivery configuration. For example, when the foil 14 is used with the attachment structure 29 of the illustrated embodiments, the mounting portion 60 of the foil 14 is slid into the slot 50, with the proximal end portion 70 first so the mounting portion of the foil is engaged between the second surface 42 of the retractor head 40 and the surface 32 of the member 30. While being slid into the slot 50, the material of the foil 14 that engages the tab 44 will be deformed and/or deflected toward the aperture 34. With continued motion of the foil 14 into the slot 50, the aperture 67 will align will with the tab 44 to provide enhanced attachment retention of the foil. FIG. 3B, for example, illustrates a foil 14 in accordance with the above described embodiments attached to the retractor head 40.

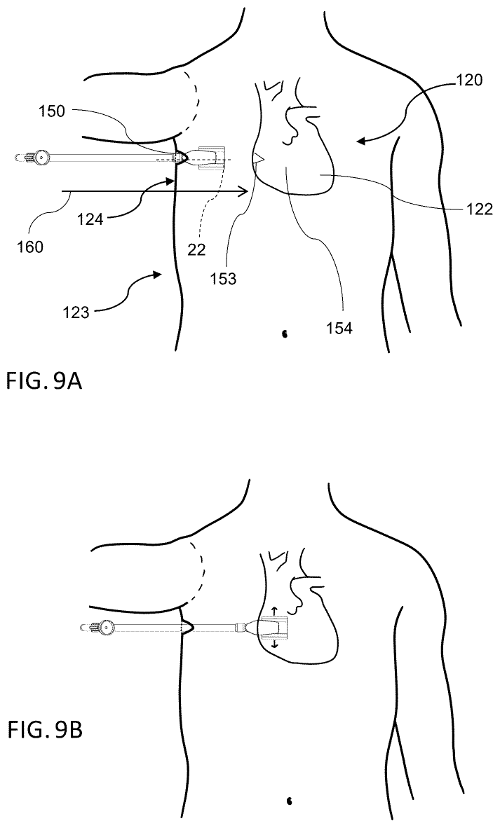

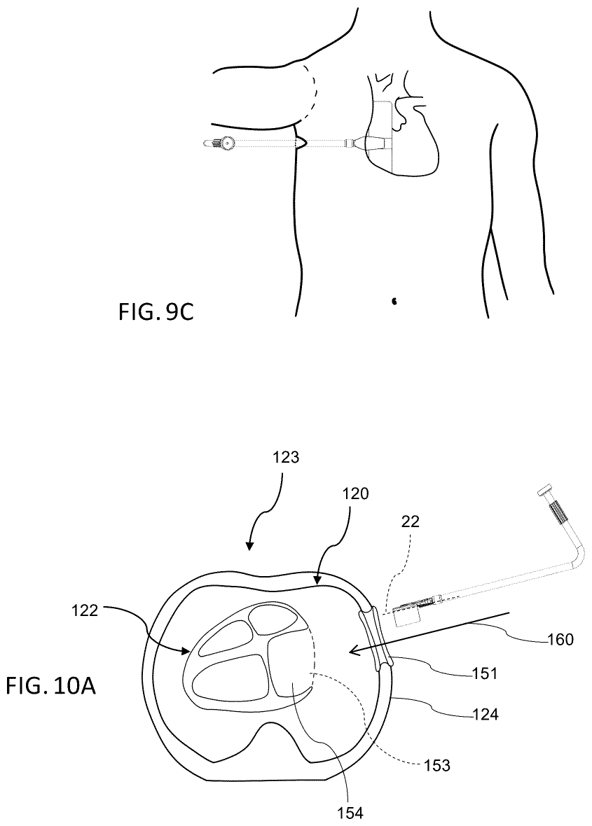

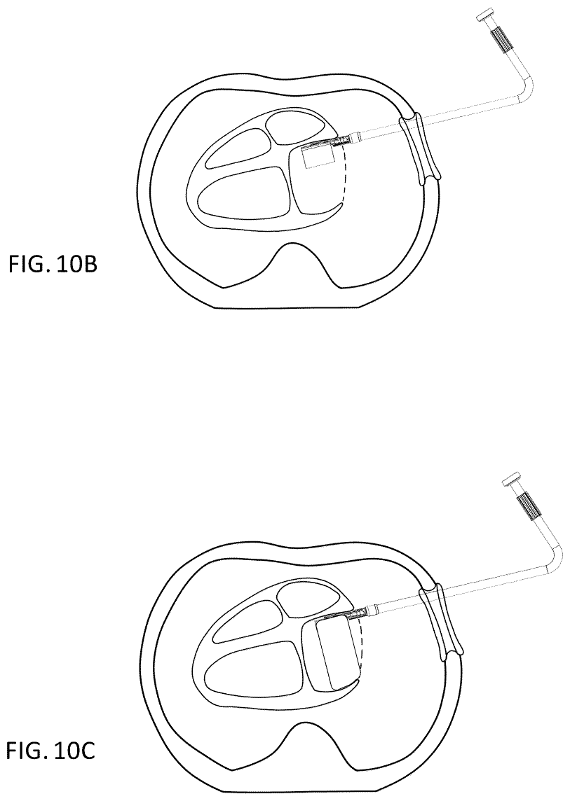

FIGS. 9A-9C and 10A-10C illustrate a medial in-line approach method of using the retractor 10 to insert the retractor head 40, and optionally a foil 14 attached to the retractor head, and to locate and position the retractor head and optional foil in a chamber of a patient's heart chamber. FIGS. 9A-9C are diagrammatic illustrations of a patient's torso 120 and heart 122, shown from the anterior (e.g., chest) side of the patient 123. FIGS. 10A-10C are diagrammatic cross sectional illustrations of the patient's torso 120 and heart 122, shown from a superior (e.g., head) side of the patient. Tissue within the accessed heart chamber, for example the right ventricle or left ventricle, can then be retracted by the retractor head 40 and optional foil 14, to provide operative access to other tissues at the surgical site, such as for example the tricuspid valve or mitral valve. Although embodiments of the method described in connection with FIGS. 9A-9C and 10A-10C include use of a foil 14 attached to the distal end portion 16 of the handle 12, for example to provide enhanced retraction stability, in other embodiments the foil is not attached to the handle or used in connection with the method. With the exception of steps involving the foil 14, embodiments of the method that do not use the foil can be substantially the same as or similar to embodiments that do involve the foil.

As shown for example in FIGS. 9A and 10A, an incision 150 may be made at a lateral side 124 of the patient 123. The incision 150 is shown at the right side of the patient 123 for purposes of example, and may be between the ribs (not shown) of the patient. The embodiments illustrated in FIGS. 10A-10C show an access port 151 inserted into the incision 150 for purposes of providing enhanced access to the operative site. An incision 153 into the chamber 154 of the heart 122 associated with the operative site, such as for example, the right ventricle of the patient 152, is made at a location on the lateral side heart. The incision 153 into the heart chamber 154 is made on a side of the heart 122 at a location medial to the incision 150. The incision 150 on the patient's side 124 and the incision 153 into the heart chamber 153 define a medial delivery axis. Incisions 150 and/or 153 may be mini-thoracotomy incisions.

The foil 14 is attached to the distal end 16 of the handle 12 in a manner such as that described above, and moved to the delivery (e.g., reduced-diameter) configuration. The retractor head 40 and foil 14 (in the delivery configuration) are then moved through the port 151 and incision 153 into the heart chamber 154, leading with the distal end portion 47 of the retractor head and the distal end portion 68 of the foil, to position the retractor head and foil in the heart chamber. The movement of the retractor head 40 and foil 14 may be done by the surgeon while grasping the handle 12. The movement is generally in a direction 160, and about a delivery path that is generally colinear with the medial delivery axis between the incisions 150 and 153. During this delivery motion the distal end axis 22 of the handle 12 is also generally colinear with the medial delivery axis. Although not shown in FIGS. 9A-9C and 10A-10C, in embodiments a surgical clamp can be used to hold the foil in the delivery configuration while the retractor head 40 and foil 14 are being inserted into the heart chamber 154. In these embodiments the surgical clamp may be inserted through the port 151 and incision 153 into the heart chamber 154 (e.g., while being generally aligned with the handle 12 of the retractor 10). The one or more tools such as a light, camera, suction or aspiration on the tool ports 100 can be used to guide and/or enhance the insertion of the retractor 10.

As illustrated by FIGS. 9B-9C and 10B-10C, after the foil 14 is positioned within the heart chamber 154 the foil can be released from the delivery configuration and expanded to an enlarged-diameter operative configuration. In embodiments that include a foil 14 formed from resilient material, the foil effectively self-expands toward its open configuration. In other embodiments other surgical instruments may be used to unfold or otherwise open the foil 14. The first surface 41 of the retractor head 40 and/or the first side surface 64 of the foil 14 may be urged into engagement with or otherwise located adjacent to the interior wall of the heart chamber 154 to retract tissues in the heart chamber and provide access to the surgical site. In embodiments (not shown), other surgical access paths into the patient's heart chamber 154 and the surgical site may be provided. For example, one or more surgical access paths may be provided through the anterior chest wall of the patient 123 in a generally posterior direction. The surgical retractor 10, with or without the foil 14, can also be used for surgical access and surgical exposure in the aorta during aortic valve surgery through an intercostal space, in embodiments.

When it is desired to remove the retractor head 40 and foil 14, the foil can be moved back to the reduced-diameter delivery configuration (for example using surgical instruments inserted into the operative site through the port 151). The retractor head 40 and foil 14 can then be withdrawn from the heart chamber 154. In embodiments, for example the retractor head 40 and foil 14 can be removed from the heart 122 by manipulating the handle 12 to move the distal end axis 22 of the handle about a path that is substantially the same as or aligned with (e.g., generally colinear with) the path used for the delivery and insertion of the retractor head 40 and foil 14.

The configuration of the retractor 10 and its use with the medial approach described above offers important advantages. For example enhanced operative visualization can be achieved (e.g., by surgical instruments inserted through the anterior chest) because the retractor is off to the lateral side. Enhanced operative visualization may also be achieved through the same main surgical incision and main working channel for the surgical instruments, hence subtracting additional surgical ports that are not needed in the chest wall. The reduced port count advantage applies not only to the atrial retractor function, but also to ports that may have been associated with tools that are incorporated into the tool port 100 of the retractor. Incorporating tools into the tool port 100 of the retractor 10 may reduce the need for other access ports and associated incisions. The configuration of the tool port 100 with respect to the retractor head 40 and foil 14 enhances the effectiveness of the tools, such as for example illumination provided by a light or visualization provided by a camera. The retractor head 40 and foil 14 can be configured for minimal if any trauma, for example by having relatively blunt edges and/or no or few holes. Enhanced surgical efficacy and outcomes can thereby be achieved by the retractor 10.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled. For example, although in some embodiments the instrument is described for use with surgical procedures on the heart, the instrument can be used in other surgical procedures, such as for example breast reconstruction procedures.

What is claimed is:

1. A surgical retractor, comprising:
a handle having a distal end portion defining a distal end axis; and
a retractor head including a retractor surface portion on the distal end portion of the handle, wherein the retractor surface portion forms an acute angle that is substantially less than 90° with respect to the distal end axis.

2. The surgical retractor of claim 1, wherein the retractor surface portion forms an angle that is less than 30° with respect to the distal end axis.

3. The surgical retractor of claim 1, wherein the retractor surface portion forms an angle that is less than 15° with respect to the distal end axis.

4. The surgical retractor of claim 1, wherein the retractor surface portion forms an angle that is less than 5° with respect to the distal end axis.

5. The surgical retractor of claim 1, wherein the retractor surface portion is parallel to the distal end axis.

6. The surgical retractor of claim 1, wherein the retractor surface portion is configured at an angle sufficiently close to parallel with respect to the distal end axis to facilitate insertion of the distal end portion of the handle into a heart chamber of a patient, optionally a left atrium or right atrium of the patient, from a side of the patient in a medial direction aligned with the distal end axis of the handle.

7. The surgical retractor of claim 1, further comprising an attachment structure on the distal end portion of the handle configured to releasably attach a foil to the retractor head, wherein the retractor head and/or the attachment structure are configured such that a surface portion of the foil, when attached to the retractor head, forms an acute angle that is substantially less than 90° with respect to the distal end axis.

8. The surgical retractor of claim 7, wherein the retractor head and/or the attachment structure are configured such that a surface portion of the foil, when attached to the retractor head, forms an angle that is less than 30° with respect to the distal end axis.

9. The surgical retractor of claim 7, wherein the retractor head and/or the attachment structure are configured such that a surface portion of the foil, when attached to the retractor head, forms an angle that is less than 15° with respect to the distal end axis.

10. The surgical retractor of claim 7, wherein the retractor head and/or the attachment structure are configured such that a surface portion of the foil, when attached to the retractor head, forms an angle that is less than 5° with respect to the distal end axis.

11. The surgical retractor of claim 7, wherein the retractor head and/or the attachment structure are configured such that a surface portion of the foil, when attached to the retractor head, is parallel to the distal end axis.

12. The surgical retractor of claim 7, wherein the retractor head and/or attachment structure are configured such that the surface portion of the foil, when attached to the retractor head, is at an angle sufficiently close to parallel with respect to the distal end axis to facilitate insertion of the distal end portion of the handle and the foil into a heart chamber of a patient, optionally a left atrium or right atrium of the patient, from a side of the patient in a medial direction aligned with the distal end axis of the handle.

13. The surgical retractor of claim 7, wherein the attachment structure comprises a tab.

14. The surgical retractor of claim 7, wherein:
the retractor head includes a first blade; and
the attachment structure comprises a second blade adjacent to the first blade, wherein the first and second blades define a slot configured to releasably receive the foil.

15. The surgical retractor of claim 14, wherein one of the first and second blades, optionally the first blade, includes a tab extending into the slot.

16. The surgical retractor of claim 15, wherein:
the other of the first and second blades, optionally the second blade, includes an opening; and
the tab extends into the opening.

17. The surgical retractor of claim 15, further comprising a foil attached to the distal end portion of the handle by the attachment structure, wherein the foil includes a foil opening and the tab extends into the foil opening.

18. The surgical retractor of claim 17, wherein the foil comprises a resilient member configured to extend between a closed or reduced diameter delivery configuration and an increased diameter open configuration

19. The surgical retractor of claim 7, further comprising a foil attached to the distal end portion of the handle by the attachment structure.

20. The surgical retractor of claim 19, wherein the foil comprises a resilient member configured to extend between a closed or reduced diameter delivery configuration and an increased diameter open configuration.

21. The surgical retractor of claim 1, further comprising a light or imaging device on the distal end portion of the handle.

22. The surgical retractor of claim 21, wherein the light or imaging device define an optical axis that extends at a non-zero acute angle with respect to the distal end axis.

23. The surgical retractor of claim 1, wherein:
the distal end portion of the handle is linear; and
the linear distal end portion of the handle has a length that is sufficiently long that the linear distal end portion can extend outside of the patient after the distal end portion and the retractor head are inserted into the patient's heart chamber.

24. A method for providing surgical access to a heart chamber of a patient, optionally a right atrium or a left atrium, by the retractor of claim 1, comprising inserting the retractor head into the heart chamber of the patient from a side of the patient at least substantially in a medial direction aligned (e.g., colinear) with the distal end axis of the distal end portion of the handle.

25. The method of claim 24, using the surgical retractor including the foil in the delivery configuration.

26. The method of claim 25, further comprising causing the foil to open to an operative position in the heart chamber and retract tissue in the heart.

27. The method of claim 23, wherein inserting the retractor head includes moving the distal end portion of the handle in a direction parallel to the distal end axis.

28. The method of claim 27, wherein inserting the retractor head includes inserting a linear portion of the handle including the distal end portion into the patient and positioning the retractor head and optionally the foil in the heart chamber while the linear portion of the handle extends outside of the side of the patient.
